(19) United States
(12) Patent Application Publication
(10) Pub. No.: US 2014/0051782 A1
(43) Pub. Date: Feb. 20, 2014

(54) PROCESS FOR PRODUCING A DENTAL FILLER

(71) Applicants: Joshua James Cheetham, Windsor (AU); Wilson Ta, Aspendale Gardens (AU); Paul James Farrar, Box Hill North (AU)

(72) Inventors: Joshua James Cheetham, Windsor (AU); Wilson Ta, Aspendale Gardens (AU); Paul James Farrar, Box Hill North (AU)

(21) Appl. No.: 13/587,903

(22) Filed: Aug. 16, 2012

(51) Int. Cl. A61K 6/00

(52) U.S. Cl. CPC A61K 6/0088
USPC 523/116; 264/121

(57) ABSTRACT

A process for producing a particulate dental filler in which a dental particulate inorganic material is admixed with a curable organic monomeric material containing acrylate groups to produce a suspension in a liquid. The suspension is spray dried to produce an atomized powder and the monomeric material is cured.

The present invention relates to a process for producing a dental filler.

Many dental compositions contain mixtures of resins and fillers such as restoratives, cements and adhesives. The fillers are typically used in the compositions to increase strength and wear resistance and to reduce polymerization shrinkage.

However, the use of filler tends to reduce polishability of the dental compositions especially where fillers of large particle size are used. Fine fillers produce better polishability but cannot be used in high loadings as their high surface areas may cause rapid thickening of the composition.

Thus, there is a need to find a compromise between fillers of large particle size and fine particle size to achieve desired results.

Attempts have been made to address this in the past but the results obtained have not been entirely satisfactory.

The present invention relates to a process for producing a dental filler in which problems encountered in the prior art are at least partially alleviated.

In accordance with one aspect of the present invention there is provided a process for producing a particulate dental filler which comprises mixing a dental particulate inorganic material with a curable organic monomer containing acrylate groups in a liquid solvent to produce a suspension, the suspension containing inorganic material particles coated with the monomer, spray drying the suspension to produce an atomized powder of the inorganic material coated with the monomer, and curing the organic component of the atomized powder.

It is found that in the spray drying step the particulate inorganic material tends to be formed into agglomerates which are bound together by the resin. The agglomerate formation produces a powder of the particulate inorganic material having particles with irregular surfaces which enhances adhesion to the organic monomer or monomeric resin in use.

Preferably, in the spray drying step of the present invention atomised droplets are produced which dry rapidly. Further, the surface tension in the liquid solvent preferably causes the droplets to form spheres which morphology is maintained in the residual solids of the droplets.

The particulate inorganic material may be coated with a silane to improve adhesion of the organic monomer to the particulate inorganic material.

The acrylate containing monomers which may be used in the present invention are ones which are capable of being dissolved in, suspended in or mixed with a solvent. Examples include acrylates and methylacrylates such as mono-, di- or poly-acrylates and methacrylates such as methyl acrylate, methyl methacrylate, ethyl acrylate, isopropyl methacrylate, n-hexyl acrylate, stearyl acrylate, allyl acrylate, glycerol diacrylate, glycerol triacrylate, ethyleneglycol diacrylate, diethyleneglycol diacrylate, triethyleneglycol dimethacrylate, 1,3-propanediol diacrylate, 1,3-propanediol dimethacrylate, trimethylolpropane triacrylate, 1,2,4-butanetriol trimethacrylate, 1,4-cyclohexanediol diacrylate, pentaerythritol triacrylate, pentaerythritol tetraacrylate, pentaerythritol tetramethacrylate, the diglycidyl methacrylate of bis-phenol A (“Bis-GMA”), and ethoxylated bis-phenol A dimethacrylates.

Preferably, the acrylate containing monomer is one which is water soluble or miscible so that the solvent used in the process of the present invention can be an aqueous solvent. This has the advantage of avoiding the need for use of potentially flammable or explosive solvents. In general, solvents which may be used in the present invention are ones which dissolve or suspend the monomer and are relatively unreactive with the components of the composition to be spray dried. Further, the solvent needs to be one which evaporates during the spray drying step. Preferred solvents useful in the present invention are water and solvents that are miscible with water. Examples of water-miscible solvents are alcohols such as methanol, ethanol, 1-propanol, 2-propanol, tert-butanol, and 1-methoxy-2-propanol, and ketones such as acetone and methyl ethyl ketone.

Whilst aqueous solvents are preferred in the process of the present invention it is possible to use non-aqueous solvents which may be flammable. In this case the spray drying step may be done in a closed loop system in which a low oxygen gas stream is used to remove solvent from the atomized material.

The use of non-aqueous solvents enables water immiscible monomers to be stabilized in the present invention. Also, faster drying times of the atomized material may be obtained by the use of non aqueous solvents.

The inorganic particulate dental materials used in the present invention may be selected from a wide range of materials. Examples of typical dental materials are pyrogenic silica, precipitated silica, X-ray opaque glasses, barium sulphate, zirconium oxide, strontium fluoride and ytterbium fluoride.

As discussed above, the particulate dental inorganic material is preferably silane-coated to improve adhesion with the curable monomeric resins. Examples of silane materials which may be used are isooctyltrimethoxysilane, phenyltrimethoxysilane, n-octyltrimethoxysilane, do decyltrimethoxysilane, octadecyltrimethoxysilane, propyltrimethoxysilane, hexyltrimethoxysilane, 3-mercaptopropyltrimethoxysilane, 3 -aminopropyltrimethoxysilane, 3 -glycidoxypropyltrimethoxysilane, 3-methacryloyloxypropyl trimethoxysilane, and combinations thereof. Water-soluble silanes are also suitable, specifically including poly(alkylene oxide) group-containing silanes such as Silquest A1230.

The curing of the organic monomer may be assisted by the use of light or thermal initiators. Examples of light-cure initiators include benzoin alkyl ethers or benzoin alkyl esters, benzyl monoketals, acyl phosphine oxides or aliphatic and aromatic 1,2-diketo compounds such as 2,2-diethoxyacetophenone 9,10-phenanthrene quinone, diacetyl 4,4′-dichlorobenzyl 4,4′-dialkoxybenzyl and camphor quinone. Photoinitiators can be used alone or in combination with a reducing agent. Examples of reducing agents are amines such as aliphatic or aromatic tertiary amines, e.g. N,N-dimethyl-p-toluidine or triethanol amine, cyanoethyl methyl aniline, trimethyl amine, N,N-dimethyl aniline, N-methyl diphenyl amine, N,N-3,5-tetramethyl aniline and 4-dimethylaminobenzoic acid ethyl ester or organic phosphites.

Camphor quinone plus ethyl-4-(N,N-dimethyl amino)benzoate, 2-(ethyl hexyl)-4-(N,N-dimethylamino) benzoate or N,N-dimethylaminoethyl methacrylate, for example, are well-established photoinitiator systems. 2,4,6-Tri-methyl benzoyl diphenyl phosphine oxide is particularly suitable as an initiator for polymerisation initiated by UV light.

Examples of thermal-initiators are peroxides (e.g., benzoyl peroxide and lauryl peroxide) and azo compounds (e.g., 2,2-azobis-isobutyronitrile).

Heat may also be used to initiate the polymerization of free radically active groups. Examples of heat sources suitable for use in the present invention include inductive, convective, and radiant heat sources. Thermal sources should be preferably capable of generating temperatures of about 40° C. and at most about 150° C. under normal conditions or at elevated pressure.

Typically, in the spray drying step the suspension produced in the process of the present invention is pumped through an atomizing nozzle into a stream of fast flowing heated gas such as air. This causes the suspension to form into atomized droplets which dry rapidly as they are carried by the gas stream because of the high surface area of the droplets formed by the atomizing process. Further, surface tension of the liquid tends to cause the droplets to form into spheres and as drying progresses this morphology is maintained by the residual solids. Thus, the resulting atomized powder typically has a mean particle size (d₅₀) of 5 to 100 microns, preferably 10 to 80 microns.

The particle size distribution of the powder may be set by controlling droplet size and the composition of the suspension. The size of the atomized droplets is essentially controlled by adjustment of the spray drying operating conditions such as nozzle type, size and configuration, inlet air temperature, outlet air temperature, atomizing pressure, fan speed and feed rate. Further, the composition of the suspension affects the characteristics of the droplets through such attributes as viscosity, surface tension and solids loading.

Curing of the spray-dried composite agglomerates coated with monomers may be achieved by any method known to those skilled in the art, such as heat-curing or light-curing. Heat-curing can be carried out at any temperature provided it does not cause damage to the resin components of the agglomerates. Typically this is between 30° C. and 200° C., but preferably between 40° C. and 150° C. Light-curing can be activated using any suitable wavelength, typically in the ultraviolet- to visible-light range (200-800nm).

Protective atmospheres can be utilised during the curing process to reduce damage to the agglomerates caused by oxidation, or to reduce the inhibition of the cure caused by the presence of oxygen. Curing atmospheres useful in the present invention are those that are inert or contain relatively nonreactive gases, such as nitrogen, helium, argon, and carbon dioxide. Alternatively, reduced pressure can be used to reduce the oxygen level during the curing process. Curing in air at atmospheric pressure is also a suitable method, provided the temperature is low enough to minimize oxidation.

The present invention will now be illustrated by the following examples.

### EXAMPLES

In the following examples the resins used are as follows.

| | | Monomer | |
|---|---|---|---|
| Ingredient | Monomer 1 | 2 | Monomer 3 |
| Bisphenol A-Glycidyl | — | — | 67.00 g |
| Methacrylate | | | |
| Polyethylene glycol 400 | 400.00 g | 30.00 g | — |
| dimethacrylate | | | |
| Tetraethylene glycol | 100.00 g | — | 33.0 g |
| dimethacrylate | | | |
| Glycol dimethacrylate | — | 20.00 g | — |
| Camphorquinone | — | — | 0.17 g |
| Ethyl-4-dimethylamino benzoate | — | — | 0.30 g |
| Benzoyl Peroxide | 1.50 g | 0.15 g | — |
| Butylated Hydroxy Toluene | 0.10 g | 0.01 g | 0.03 g |
| Total | 501.60 g | 50.16 g | 100.50 g |

### Example 1

5400 g de-ionised water was added to a mixing vessel fitted with an overhead stirrer. 2700 g 0.4 micron Barium glass was slowly added to the stirred water. A mixture of 300.0 g “Monomer 1” and 265.0 g 3-methacryloxypropyl trimethoxysilane was added slowly to the stirred slurry. Stirring was continued until the slurry had thickened to a stable consistency. The mixture was spray-dried to produce a fine white powder using the following conditions:

| | |
|---|---|
| Inlet Temp (° C.) | 210 |
| Outlet Temp (° C.) | 95-100 |
| Feed Rate (ml/min) | 100 |
| Atomising Pressure | ~3 bar |
| Nozzle Diameter | 2.5 mm |

1500 g of the resulting spray-dried powder was heated in a vacuum oven to 100° C. for 3 hours with a nitrogen atmosphere. The heat-cured powder was sieved through 150 micron mesh. Results are shown in Table 1.

### Example 2

200 g de-ionised water was added to a beaker fitted with a magnetic stirrer. 76.0 g 0.7 micron strontium fluoroaluminosilicate glass was slowly added to the stirred water. A mixture of 3.0 g 3-methacryloxypropyl trimethoxysilane and 1.0 g poly (alkylene oxide) silane was added slowly to the stirred slurry. Stirring was continued until the slurry had thickened to a stable consistency.

Separately, 25.0 g colloidal silica (40% solids, 20 nm, NH₃ stabilized) was charged to a small plastic container, fitted with a magnetic stirrer. A mixture of 28.13 g propylene glycol methyl ether and 0.75 g

3-methacryloxypropyl trimethoxysilane was added slowly to the stirred colloidal silica. A cap was fitted to the container to seal the contents. The stirrer/hotplate was set to 80° C. and the mixture was stirred for 24 hours. The silane-treated colloidal silica solution was allowed to cool to room temperature. 10.0 g “Monomer 2” was added slowly to 50.0 g of the stirred silane-treated colloidal silica. This was then added slowly to the glass/silica slurry. The resulting mixture was spray-dried to form a fine white powder. The following conditions were used:

| | |
|---|---|
| Inlet Temp (° C.) | 220 |
| Outlet Temp (° C.) | 100-115 |
| Feed Rate (ml/min) | 9 |
| Atomising Pressure | ~5 bar |
| Nozzle Diameter | ~1.0 mm |

80 g spray-dried powder was heated in a vacuum oven to 70° C. for 16 hours with a nitrogen atmosphere. The heat-cured powder was sieved through 90 micron mesh. Results are shown in Table 1.

TABLE 1

| | Example 1 | Example 2 |
|---|---|---|
| Density | 2.51 | 2.54 |
| (g/cm³) | | |
| Particle Size | d10: 8.0 micron | d10: 6.2 micron |
| Distribution | d50: 23.7 micron | d50: 13.1 micron |
| | d90: 62.6 micron | d90: 26.6 micron |

### Example 3

A composite paste was made according to the formulation in Table 2 and the results of testing are shown in Table 3.

TABLE 2

| Ingredient | Example 3 |
|---|---|
| Monomer 3 | 12.25 g |
| Silane treated fumed silica | 3.25 g |
| 0.7μ Barium Glass | 24.00 g |
| Pre-polymerised Filler | 18.00 g |
| (Example 1) | |

TABLE 3

| Property | Example 3 |
|---|---|
| Radiopacity (% Al) | 300 |
| % Translucency | 52.1 |
| Flexural Strength (MPa) | 118.6 |

It has been found that the filler of the present invention provides improvements over fillers produced by current state of the art methods. The filler of the present invention has a high inorganic particle loading which leads to composite material with higher radiopacity and better controlled aesthetic properties. The tortuous surface, formed by the agglomerates also produces a composite material with better mechanical strength

Modifications and variations as would be apparent to a skilled addressee are deemed to be within the scope of the present invention.

1. A process for producing a particulate dental filler which comprises mixing a dental particulate inorganic material with a curable organic monomer containing acrylate groups in a liquid solvent to produce a suspension, the suspension containing inorganic material particles coated with the monomer, spray drying the suspension to produce an atomized powder of the inorganic material coated with the monomer, and curing the organic component of the atomized powder.

2. A process according to claim 1, wherein the particulate inorganic material tends to be formed into agglomerates which are surrounded by the resin, such that the atomized powder has particles with irregular surfaces.

3. A process according to claim 1, wherein the particles of the atomized powder are generally spherical in overall shape.

4. A process according to claim 1, wherein the particulate inorganic material is coated with silane to improve adhesion of the monomer to the particulate inorganic material.

5. A process according to claim 1, in which the solvent is aqueous.

6. A process according to claim 1, wherein in the spray drying step the suspension is pumped through an atomizing nozzle into a stream of fast flowing heated gas so that the suspension forms atomized droplets which dry as they are carried by the gas stream so as to form inorganic particles coated with the monomer.

7. A process according to claim 1, wherein the atomized powder has a mean particle size of 5 to 100 microns.

8. A process according to claim 7, wherein the atomized power has a mean particle size of 10 to 80 microns.

9. A particulate dental filler when produced by the process of claim 1.
